SEQUENCE LISTING (6 sequences)

SEQ ID NO: 1 - DNA, 30 bp, Artificial
  probe/primer
cctaggatccaagagggcaaatgtgcaaag

SEQ ID NO: 2 - DNA, 30 bp, Artificial
  probe/primer
gaaagggaaacagagggaattcagatgttc

SEQ ID NO: 3 - DNA, 30 bp, Artificial; identical to SEQ ID NO: 1
  probe/primer

SEQ ID NO: 4 - DNA, 37 bp, Artificial
  probe/primer
cctgcataagtaaacttctcaataaagcgtggtattc

SEQ ID NO: 5 - DNA, 38 bp, Artificial
  probe/primer
tatagatcttggccccagcgcgcacgggcgatggcgaa

SEQ ID NO: 6 - DNA, 29 bp, Artificial
  probe/primer
tatagatctgatgcagaaggcaacagcag

(19) United States
(12) Patent Application Publication
(10) Pub. No.: US 2004/0185548 A1
(43) Pub. Date: Sep. 23, 2004

(54) Hybrid receptors for efficient assay of modulators of receptor protein-tyrosine kinases

(76) Inventor: Qun-Sheng Ji, Babylon, NY (US)

Correspondence Address: Alexander A. Stewart, OSI Pharmaceuticals, Inc., Suite 110, 58 South Service Road, Melville, NY, 11747, US

(21) Appl. No.: 10/801,868

(22) Filed: Mar. 16, 2004

Related U.S. Application Data
(60) Provisional application No. 60/455,378, filed on Mar. 17, 2003.

(51) Int. Cl. C12N 9/12

(52) U.S. Cl. 435/194; 514/12

(57) ABSTRACT

Hybrid receptors are provided that comprise (a) the extracellular domain of the Ret receptor kinase, containing one or more amino acid residue substitutions, deletions or additions that render it capable of activating an intracellular receptor kinase domain in a ligand-independent manner, and (b) the kinase domain of a heterologous receptor protein kinase. The present invention is also directed to nucleic acids and expression vectors encoding these hybrid receptor proteins, host cells expressing these hybrid receptor proteins, methods for detecting a modulator of receptor protein kinase activity, and membrane preparations comprising recombinantly produced hybrid receptor protein. The hybrid receptors are useful for assays for the determination of modulators of receptor protein kinase activity, being particularly useful in cases where the ligand for the receptor kinase is unknown, or difficult to obtain or use.

### CROSS-REFERENCE TO RELATED APPLICATIONS

This application claims the benefit of U.S. Provisional Application No. 60/455378, filed Mar. 17, 2003, which is herein incorporated by reference in its entirety.

### STATEMENT REGARDING FEDERALLY SPONSORED RESEARCH OR DEVELOPMENT

Not applicable.

### REFERENCE TO SEQUENCE LISTING

A complete sequence listing section is included herein.

### BACKGROUND OF THE INVENTION

This invention is directed to in vitro methods for screening candidate drugs for their ability to modulate the activity of a receptor protein-tyrosine kinase.

It is known that a cell may become cancerous by virtue of the transformation of a portion of its DNA into an oncogene (i.e. a gene which, on activation, leads to the formation of malignant tumor cells). Many oncogenes encode proteins that are aberrant protein-tyrosine kinases capable of causing cell transformation. These kinases function by catalyzing the transfer of the γ phosphate of ATP to the hydroxyl group of the tyrosine on target proteins. Alternatively, the overexpression of a normal proto-oncogenic tyrosine kinase may also result in proliferative disorders, sometimes resulting in a malignant phenotype. It is known that such kinases are frequently aberrantly expressed in common human cancers.

Accordingly, it has been recognized that inhibitors of protein-tyrosine kinases are useful as selective inhibitors of the growth of mammalian cancer cells (de Bono J. S. and Rowinsky, E. K. (2002) Trends in Mol. Medicine 8:S19-S26; Dancey, J. and Sausville, E. A. (2003) Nature Rev. Drug Discovery 2:92-313). For example, Gleevec™ (also known as imatinib mesylate, or STI571), a 2-phenylpyrimidine tyrosine kinase inhibitor that inhibits the kinase activity of the BCR-ABL fusion gene product, was recently approved by the U.S. Food and Drug Administration for the treatment of CML. This compound, in addition to inhibiting BCR-ABL kinase, also inhibits KIT kinase and PDGF receptor kinase, although it is not effective against all mutant isoforms of KIT kinase. In recent clinical studies on the use of Gleevec™ to treat patients with GIST, a disease in which KIT kinase is involved in transformation of the cells, many of the patients have shown marked clinical improvement. Other kinase inhibitors show even greater selectively. For example, the 4-anilinoquinazoline compound Tarceva™ inhibits only EGF receptor kinase with high potency, although it can inhibit the signal transduction of other receptor kinases, probably by virtue of the fact that these receptors heterodimerize with EGF receptor.

Although such anti-cancer compounds make a significant contribution to the art, there is a continuing search in this field of art for improved anti-cancer pharmaceuticals with better selectivity or potency, reduced toxicity, or fewer side effects. There is also a continuing need for improvements in methods for finding such pharmaceuticals, including assay systems that are simpler, more reproducible, more efficient, more environmentally friendly, more amenable to high-throughput screening, or less expensive.

One type of tyrosine kinase for which selective inhibitors continue to be sought are receptor tyrosine kinases. These are large enzymes that typically span the cell membrane and possess (a) an extracellular binding domain for a ligand, such as a growth factor, (b) a transmembrane domain that is a highly hydrophobic region of about 20 to 25 residues and is responsible for embedding the receptor in the cell membrane, and (c) an intracellular portion which contains a conserved protein-tyrosine kinase domain, and additional regulatory sequences that are subjected to autophosphorylation and phosphorylation by heterologous protein kinases (Schlessinger, J. (2000) Cell 103:211-225). Binding of ligand typically results in receptor homodimerization, activation of tyrosine kinase activity, and subsequent phosphorylation of a variety of protein substrates, typically including the receptor molecule itself. Many of such phosphorylated proteins are effectors of intracellular signal transduction, frequently leading to enhanced cell proliferation. With some receptor kinases, receptor heterodimerization can also occur (Lemmon, M. A. and Schlessinger, J. 1994, TIBS, 19:459-463). Receptor tyrosine kinases play an important role in the control of most fundamental cellular processes including cell proliferation, migration, survival, differentiation, as well as the cell cycle and metabolism.

Receptor tyrosine kinases are the largest group of dominant oncogenes with structural homology. Enhanced or ligand-independent constitutive kinase activity for such kinases associated with common human cancers results from either overexpression of the kinase, or gain-of-function mutations and deletions (Robertson, S. C. et. al. 2000, Trends in Genetics, 16: 265-271). One such kinase is the Ret receptor tyrosine kinase, whose function is essential for development of the kidney and enteric system, and for neuronal differentiation and survival. Germline gain-of-function mutations in Ret are involved in three family tumor syndromes: multiple endocrine neoplasia 2A (MEN2A), MEN2B, and familial medullary thyroid carcinoma (MTC) (Jhiang, S. M., 2000, Oncogene 19:5590-5597; Santoro, M. et. al., 2002, Ann. N.Y. Acad. Sci. 963:116-121; Altanerova, V., 2001, Neoplasma 48:325-331). Almost 100% of patients with MEN2A and MTC have mutations that affect one of six juxtamembrane cysteines (Cys609, 611, 618, 620, 630 and 634) in the Ret extracellular domain. These mutations result in the substitution of a cysteine with a different amino acid. This leads to subsequent ligand-independent kinase activation, caused by formation of intermolecular disulphide bonds between Ret molecules, and constitutive dimerization.

Many receptor tyrosine kinases have been identified that have an in vitro assayable activity that is dependent upon ligand interaction. For example, the binding of EGF to the epidermal growth receptor stimulates the kinase, or phosphotransferase, domain in the receptor to phosphorylate certain target amino acid residues located in its intracellular cytoplasmic domain, i.e. autophosphorylation. Unfortunately, for other receptors there is no known ligand, it is difficult to quantitatively assay ligand-dependent activation, or the ligand is difficult to obtain or use. Nevertheless, it is often desirable for therapeutic purposes to identify modulators of the kinase activity of such receptors, particularly inhibitors. It would be highly desirable to find a method for screening candidate drugs for such receptors that does not require use of a ligand, and can furthermore determine the activity of such drug candidates in a cellular environment, comparable to that likely to be encountered in therapeutic use in vivo. In order to facilitate screening of candidate drugs for modulators of receptor protein-tyrosine kinase activity, the invention described herein provides such a method.

### SUMMARY OF THE INVENTION

This invention provides novel hybrid receptors that comprise (a) the extracellular domain of the Ret receptor kinase, containing one or more amino acid residue substitutions, deletions or additions that render it capable of activating an intracellular receptor protein-tyrosine kinase domain in a ligand-independent manner, and (b) the kinase domain of a heterologous receptor protein-tyrosine kinase. The heterologous receptor protein-tyrosine kinase domain of the hybrid receptor is rendered in an active conformation by its association with the modified Ret extracellular domain. The present invention is also directed to nucleic acids and expression vectors encoding these hybrid receptor proteins, host cells expressing these hybrid receptor proteins, methods for detecting a modulator of receptor protein kinase activity, and membrane preparations comprising recombinantly produced hybrid receptor protein.

The hybrid receptors of this invention are particularly useful for in vitro cellular assays for the determination of modulators of receptor protein kinase activity, being especially useful in cases where the ligand for the receptor kinase is unknown, or difficult to obtain or use. For example, the hybrid receptors are useful in in vitro cellular screening methods for identifying or characterizing inhibitors of receptor protein kinases. A particular advantage of the hybrid receptor of this invention is that it enables a universal, portable assay system for determining the activity of inhibitors of any receptor kinase with an intracellular protein-tyrosine kinase domain.

### BRIEF DESCRIPTION OF THE FIGURES

FIG. 1 Schematic composition of a construct for expression of chimeras of the Ret containing cysteine 634 mutation and Tie2 receptors. The region coding for different receptor mutants is indicated. The restriction sites used for plasmid constructions are also shown.

FIG. 2 Ligand-independent tyrosine phosphorylation of Ret^{C634W}/Tie2 chimeric receptors. NIH 3T3 cells transiently expressing Ret^{C634W}/Tie2^{WT} or Ret^{C634W}/Tie2^{ΔC} were treated with or without 1 mM Na₃VO₄ for the indicated period. (A) Cell lysates prepared from above cells were precipitated with anti-Ret extracellular domain, and the precipitates were subsequently probed by Western blotting for phosphotyrosine. Results from two independent experiments are shown. (B) The same cells were examined by Western blotting for their expression of Ret^{C634W}/Tie2^{WT} and Ret^{C634W}/Tie2^{ΔC}. In both panels, bound antibody was detected by ECL (see Experimental Details).

FIG. 3 In vitro kinase activity of Ret^{C634W}/Tie2 chimeric receptors. Equal amounts of immunoprecipitates from NIH 3T3 cells transiently expressing Ret^{C634W}/Tie2^{WT} or Ret^{C634W}/Tie2^{ΔC} were used to phosphorylate an exogenous substrate polyGlu-Tyr that was pre-coated in a 96-well plate. Expression of the chimeric receptors was verified by Western blot (not shown). Tyrosine phosphorylated polyGlu-Tyr was quantified with an HRP-conjugated phosphotyrosine antibody and ABTS (see Experimental Details). Similar results were obtained in two independent experiments.

FIG. 4 Inhibtion of autophosphorylation of Ret^{C634W}/Tie2 chimeric receptor. NIH 3T3 cells transiently expressing Ret^{C634W}/Tie2^{ΔC} were treated with or without two Tie2 antagonists at the indicated concentrations at 37° C. for 2 hours. Cell lysates prepared from these cells were precipitated with anti-Ret extracellular domain, and the precipitates were subsequently probed by Western blotting for phosphotyrosine. The bound antibody was detected by ECL. Similar results were obtained in three independent experiments.

FIG. 5 IC50 determination of Tie2 antagonist in a cell-based autophosphorylation assay. NIH 3T3 stable cells expressing Ret^{C634W}/Tie2^{ΔC} were plated in a 6-well plate, and treated with or without a Tie2 antagonist at the indicated concentrations at 37° C. for 2 hours. The cells were lysed and the lysates were parallel transferred to a pre-coated anti-Ret 96-well plate. After incubation at 4° C. overnight, the tyrosine phosphorylation was quantified with an HRP-conjugated phosphotyrosine antibody and the Femto maximum sensitivity substrate (see Experimental Details). The IC50 curves were plotted by an ExcelFit program. The results were expressed as the mean of duplicate samples for each concentration of compound. IC50 curves from two independent assays are shown.

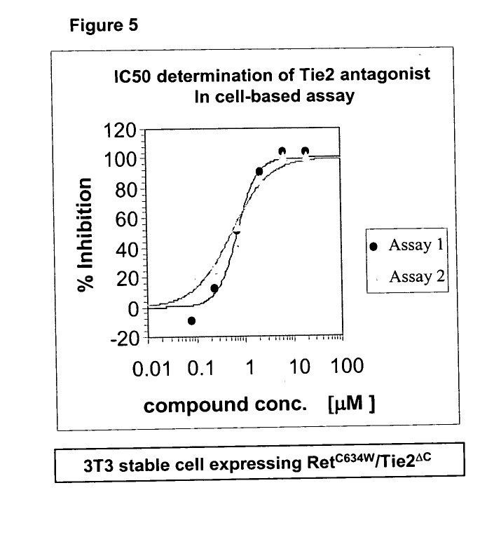

### DETAILED DESCRIPTION OF THE INVENTION

This invention provides novel hybrid receptors that comprise (a) a modified extracellular domain of the Ret receptor kinase, containing one or more amino acid residue substitutions, deletions or additions that render it capable of activating an intracellular receptor protein-tyrosine kinase domain in a ligand-independent manner, and (b) the kinase domain of a heterologous receptor protein-tyrosine kinase. The heterologous receptor protein-tyrosine kinase domain of the hybrid receptor is rendered in an active conformation by its association with the modified Ret extracellular domain.

The present invention is also directed to nucleic acids and expression vectors encoding these hybrid receptor proteins, host cells expressing these hybrid receptor proteins, methods for detecting a modulator of receptor protein kinase activity, and membrane preparations comprising recombinantly produced hybrid receptor protein.

The term “ligand-independent” as used herein, as for example applied to receptor kinase activity, refers to activity expressed by an enzyme that is not dependent on the presence of a receptor ligand. A receptor having ligand-independent kinase activity will not necessarily preclude the binding of ligand to that receptor to produce additional activation of the kinase activity. The nature of the receptor and of any modifications determine the extent of ligand-independence. In the latter case the ligand-independent activity can be thought of as a high constitutive level of kinase activity.

The term “constitutive” as used herein, as for example applied to receptor kinase activity, refers to activity expressed by an enzyme that is not dependent on the presence of a receptor ligand, or other activating molecules. In other words, constitutive refers to that part of the maximum activity of the enzyme that is always expressed, regardless of the presence of activating moieties. Depending on the nature of the enzyme molecule, all of the activity of an enzyme may be constitutive, or the enzyme may be further activated by the binding of other molecules (e.g. ligands).

The terms “hybrid” or “chimeric”, as used herein, as for example applied to a receptor protein kinase, both refer to a macromolecular fusion that is comprised of different components, two or more of which originate from different species or from different genes. The two terms commonly are used interchangeably in the art.

The hybrid receptors of this invention can be used in the form of recombinantly expressed proteins in cells, as membrane preparations prepared from such cells, or as purified receptor proteins prepared from these cells, cell membranes or conditioned medium of such cells in cases where the receptor protein is secreted from the cell. Thus, in assays that involve incubating the hybrid receptor with a test sample suspected to contain a modulator of the hybrid receptor protein-tyrosine kinase activity, the receptor is added as a cell preparation, a membrane preparation, or an isolated protein. In cellular assays, cells are preferably added as a monolayer or suspension cell culture.

The hybrid receptors of this invention are useful for in vitro cellular assays for the determination of modulators of receptor protein kinase activity that are potential drug candidates, being particularly useful in cases where the ligand for the receptor kinase is unknown, or difficult to obtain or use. They are also useful for determining the effects of a kinase modulator on a group or array of kinases in order to determine the compound's selectivity. In the latter case, all such assays can be performed in the same cell background with no need to add different activating ligands for each kinase. This will improve screening efficiency where large numbers of assays are required, and will negate any technical problems associated with the use of the receptor ligands.

The hybrid receptor of this invention is particularly useful in in vitro cellular screening methods for identifying or characterizing inhibitors of receptor protein-tyrosine kinases. By monitoring the effect of such an inhibitor in a cellular environment, comparable to that likely to be encountered in therapeutic use in vivo, one can more readily assess the potential usefulness of the inhibitor as a drug candidate. The assays of this invention have advantages over other in vitro assays utilizing for example the soluble kinase domain of a receptor protein kinase, in that the assays of the present invention also assess the ability of the drug candidate to cross the cell membrane, and its stability in a cellular environment. One incubates the hybrid receptor with the candidate drug and assays for inhibitory activity by for example monitoring the autophosphorylation activity of the receptor kinase. A particular advantage of the hybrid receptor is that it enables a universal, portable assay system for determining the activity of inhibitors of any receptor kinase with an intracellular protein-tyrosine kinase domain.

In the practice of this invention, suitable modified extracellular domains of the Ret receptor kinase are selected from any of several extracellular Ret domains that have been described in the scientific literature as possessing amino acid substitutions, deletions or additions that confer constitutive, ligand-independent, protein-tyrosine kinase activity on the Ret receptor (e.g. see Jhiang, S. M., 2000, Oncogene 19:5590-5597; Santoro, M. et. al., 2002, Ann. N.Y. Acad. Sci. 963:116-121; Robertson, S. C. et. al. 2000, Trends in Genetics, 16: 265-271; Arlt, D. H. et. al., 2000, Oncogene, 19:3445-3448; Bongarzone, I. et. al., 1999, Oncogene 18:4833-4838; Rizzo, C. et. al., 1996, J. Biol. Chem. 46:29497-29501; Mograbi, B. et. al., 2001, Mol. Cell. Biol. 21:6719-6730; Segouffin-Cariou, C., 2000, J. Biol. Chem. 275:3568-3576; Asai, N., et. al. 1995, Mol. Cell. Biol. 15:1613-1619; Santoro, M., et. al. 1995, Science 267:381-383; Iwashita, T., et. al. 1996, Hum. Mol. Genet. 5:1577-1580; Dhappuis-Flament, S., 1998, Oncogene 17:2851-2861; Altanerova, V., 2001, Neoplasma 48:325-331; Kalinin, V. and Frilling, A. 1998, J. Mol. Med. 76:365-367), or additional domains that may be determined in the future to possess such activity. Such extracellular domains of the Ret receptor kinase, containing one or more amino acid residue substitutions, deletions or additions that render it capable of activating the Ret intracellular receptor protein-tyrosine kinase domain in a ligand-independent manner, will in the hybrid receptor of this invention render the heterologous receptor protein kinase domain in an active conformation. Typically, such ligand-independent extracellular domains of the Ret receptor kinase as described in the scientific literature, and modified extracellular domains of the Ret receptor kinase that are suitable in the practice of this invention, will contain one or more intramolecularly unpaired cysteine residues that are available for the formation of active Ret dimers via the formation of intermolecular covalent bonds (i.e. cystine, the disulphide product of two cysteines).

Amino acid residue substitutions, deletions or additions that will produce a modified Ret extracellular domain suitable for practice of this invention include, but are not limited to: (a) amino acid residue substitutions, deletions or additions that affect one or more of the six juxtamembrane cysteines (Cys 609, 611, 618, 620, 630 and 634) in the Ret extracellular domain, leading to an unpaired cysteine that can link with an unpaired cysteine in other Ret molecules, (b) replacement of one of the six juxtamembrane cysteines (Cys 609, 611, 618, 620, 630 and 634) in the Ret extracellular domain with an alternative amino acid (c) Addition, insertion or duplication of, one or more amino acid residues in the Ret extracellular domain, one of which is a cysteine residue, (d) one or more amino acid substitutions, one of which is C634W, C634R, C634Y, C634F, C634G, C634S, C630F, C634W, C620F, C618F, C620S, C618S, C620G, C618G, C611G, C611W, C620R, C618R, C609R, C620Y, C618Y, C611Y, or C609Y, in the human Ret extracellular domain, (e) substitution C634R/A640G in the human Ret extracellular domain, (f) L633, E632/L633 or residue 592-607 (16 residue) deletions in the human Ret extracellular domain, (g) insertion of the peptide HELC between residues C634 and R635, or insertion of the peptide CRT between residues L633 and C634, both in the human Ret extracellular domain, and (h) substitution C634R combined with deletion of E632/L633 in the human Ret extracellular domain.

Typically, the amino acid substitutions, deletions or additions described in the scientific literature that confer constitutive, ligand-independent, protein-tyrosine kinase activity on Ret kinase refer to modifications of the human Ret molecule. However, in the practice of this invention, comparable or corresponding amino acid substitutions, deletions or additions in the Ret kinase of other species can also be used in the hybrid receptor of this invention. The exact position of such modifications in the Ret sequence of other species can be readily determined by a comparison of the sequence of the Ret for the other species with that for human Ret using any of the many computer programs available for identifying sequence homology in proteins (e.g. see Altschul, S. F., et. al. (1997), “Gapped BLAST and PSI-BLAST: a new generation of protein database search programs”, Nucleic Acids Res. 25:3389-3402).

In the practice of this invention, suitable amino acid substitutions, deletions or additions that confer constitutive, ligand-independent, protein-tyrosine kinase activity on the hybrid receptor is understood to mean any such amino acid substitutions, deletions or additions applied singly or in combination (i.e. two or more amino acid changes selected from substitutions, deletions and additions) that leads to constitutive, ligand-independent, protein-tyrosine kinase activity in the hybrid receptor.

In the practice of this invention, suitable amino acid residue substitutions, deletions or additions that render the extracellular domain of a Ret receptor kinase capable of activating the Ret intracellular receptor protein-tyrosine kinase domain in a ligand-independent manner, and which will in the hybrid receptor of this invention render the heterologous receptor protein kinase domain in an active conformation, are readily determined by the construction of a hybrid receptor as described herein, and comparison of the properties of an unsubstituted hybrid receptor with those of a hybrid receptor with amino acid residue substitutions, deletions or additions. A measurable increase in receptor activity as a result of amino acid substitution, deletion or addition will identify suitable amino acid residue substitutions, deletions or additions that render the extracellular domain of a Ret receptor kinase capable of activating the Ret intracellular receptor protein-tyrosine kinase domain in a ligand-independent manner, and thus which will in the hybrid receptor of this invention render the heterologous receptor protein kinase domain in an active conformation. Methods for introducing such amino acid substitutions, deletions or additions into recombinant proteins are well known in the art and routinely performed, e.g. site-directed mutagenesis.

In the practice of this invention, extracellular domains from other receptor protein-tyrosine kinases that have the property of conferring constitutive or ligand-independent activity may be used to substitute for the extracellular domain of a Ret receptor kinase that is capable of activating the heterologous intracellular receptor protein-tyrosine kinase domain in a ligand-independent manner, and will in the hybrid receptor of this invention render the heterologous receptor protein kinase domain in an active conformation. Such extracellular domains include, but are not limited to, those of Tpr-MET, TEL-PDGFR, TRK-T1 and NPM-ALK receptor protein-tyrosine kinases, and of certain erbB2 and erbB4 mutant receptors (e.g. Rodrigues, G. A. and Park, M., 1993, Mol. Cell. Biol. 13:6712-6722; Jousset, C., et. al. 1997, EMBO J., 16:69-82; Greco, A., et. al. 1992, Oncogene, 7:237-242; Greenland, C., et. al. 2001, Oncogene, 20:7386-7397; Penington, D. J. et. al. 2002, Cell Growth and Differentiation, 13:247-256). Similarly, in the practice of this invention, suitable amino acid residue substitutions, deletions or additions that render the extracellular domain of other receptor protein-tyrosine kinases capable of activating their intracellular receptor protein-tyrosine kinase domains in a ligand-independent manner, and which will in the hybrid receptor of this invention render the heterologous receptor protein kinase domain in an active conformation, can be utilized. In one embodiment of this invention, the ligand-independent receptor protein-tyrosine kinase extracellular domain is any ligand-independent receptor protein-tyrosine kinase extracellular domain that comprises one or more intramolecularly unpaired cysteine residues that are available for the formation of active dimers via the formation of intermolecular covalent bonds (i.e. cystine, the disulphide product of two cysteines). In another embodiment of this invention, the ligand-independent hybrid receptor protein-tyrosine kinase extracellular domain is any extracellular domain that mediates dimerization of the hybrid receptor. In one embodiment of the latter, the ligand-independent hybrid receptor protein-tyrosine kinase extracellular domain comprises a leucine zipper motif that mediates dimerization of the hybrid receptor (e.g. Rodrigues, G. A. and Park, M., 1993, Mol. Cell. Biol. 13:6712-6722; Greco, A., et. al. 1992, Oncogene, 7:237-242; Santoro, M. M. et. al., 1996, Mol. Cell. Biol., 16:7072-7083).

In the practice of this invention, a suitable kinase domain for the heterologous receptor protein-tyrosine kinase domain of the hybrid receptor of this invention is selected from any member of the family of receptor protein-tyrosine kinases, including known, or well characterized enzymes, predicted kinase domains from family members identified by virtue of their sequence homology, or from additional members of this family yet to be discovered. Such kinase domains include, but are not limited to those of the following receptors: EGFR (HER1), HER2 (ErbB2), HER3 (ErbB3), HER4 (ErbB4), insulin receptor, IGF-1 receptor, IRR, PDGFR-alpha, PDGFR-beta, CSF-1 receptor (c-fms), KIT (SCF receptor), FLK2, FLK1, FLT4, FGFR1, FGFR2, FGFR3, FGFR4, CCK4, MET (HGF-R), RON, VEGFR1, VEGFR3, TrkA, Eph, AXL, MER, SKY (Rse), EphA2 (Eck), EphA1, EphA3, EphA4, EphA5, EphA6, EphA7, EphA8, EphB1, EphB2, EphB3, EphB4, EphB5, EphB6, RYK, Flt-3, FLT-1, TRKC (NGF receptor), TRKA, TRKB, Nck-alpha, Spry, KDR (VEGFR2), PDGF-R-alpha, Syk, Blk, FGFR-3, LTK, TIE, TEK (TIE2, angiopoietin receptor), human Tie2, ROR, DDR1, DDR2, Ret (GDNF receptor), ROS, LTK, ALK, ROR2, ROR1, RTK106, LMR1, LMR2, LMR3, KLG, RYK, MuSK, LET-23, DAF-2, F59F3.1, F59F3.5, F40G9.13, EGL-15, KIN15, KIN16, TKR-1, C08H9.8, F59F5.3, M01B2.1, R09D1.12, R09D1.13, T01G5.1, T17A3.8, W04G5.6C, W04G5.6N, Y50D4B-4, ZK938.5, B0198.3, F54F7.5, VAB-1, C16B8.1, F11D5.3, C25F6.4, C16D9.2, CAM-1, T10H9.2, B0252.1, F11E6.8, F40A3.5, R151.4, T148.1, T22B11.3, Y38H6C.20, C24G6.2A, F08F1.1, F09A5.2, and F09G2.1. The complete sequences of these proteins and their encoding DNAs from multiple species are available in public databases, e.g. Genbank.

A putative protein-tyrosine receptor may have been identified but its ligand in vivo remains unknown. For example, study of endocrine tissues from such glands as the pituitary or adrenals will lead to the identification of membrane bound proteins that are structurally similar to other known receptors, i.e. they will have a large (typically >500 residues) extracellular domain, a hydrophobic transmembrane sequence and a carboxy-terminal cytoplasmic region containing a domain with substantial homology to known protein-tyrosine kinases, and thus identified as a putative protein-tyrosine kinase. Similarly, putative receptors may be identified on malignant cells, that may be associated with the transformed phenotype. The kinase domains of such receptors are also useful in the practice of this invention, and will enable the identification of potential drug candidates that act to modulate the activity of such receptors. Such compounds will have utility not only as therapeutic agents, but also as tools to assist in the further elucidation of the biological roles of such receptors, identification of the biochemical pathways by which they act, and thus identification of potential additional targets for therapeutic intervention.

The protein-tyrosine kinase domain of the hybrid receptor protein-tyrosine kinase of this invention is heterologous to the modified extracellular domain of the Ret receptor kinase and is any kinase domain that is capable of activation in a ligand-independent manner by the modified extracellular domain of the Ret receptor kinase. This activation is generally detected by a change in the enzymatic activity or immunological identity of the kinase domain of the receptor protein-tyrosine kinase. In the practice of this invention, it is not necessary to use the entire cytoplasmic domain from a heterologous receptor protein-tyrosine, or receptor analogue, only that portion necessary to perform the desired function herein. It is well known in the art how to identify those regions of protein-tyrosine kinases that are sufficient for expression of kinase or phosphotransferase activity. It is also not necessary to use a heterologous cytoplasmic domain that is an intact, unmodified sequence from another receptor. For example, an amino acid sequence variant or derivative of the cytoplasmic domain of the receptor supplying the kinase domain is also acceptable. In one embodiment of this invention the human Tie2 intracellular domain (Tie2^{WT}, 770-1123) or a C-terminal 16 amino acid deletion form of the human Tie2 intracellular domain (Tie2^{ΔC}, 770-1107) is used.

The use of the hybrid receptors of the invention described herein harnesses the signal transducing mechanism of receptors, wherein the the conformational changes conferred by the amino acid substitutions, deletions or additions in the modified extracellular Ret domain are transduced through the receptor molecule to the kinase domain by conformational changes and intermolecular associations (e.g. dimerization) of the molecule, which changes affect the function or character of the cytoplasmic protein-tyrosine kinase domain of the heterologous receptor. It is well known from previous studies on chimeric receptor protein-tyrosine kinases that this transducing mechanism functions whether the kinase domain is homologous or heterologous to the extracellular domain, and also operates effectively even when the two domains are from different receptor protein-tyrosine kinase families (e.g. U.S. Pat. No. 4,859,609; Pandiella, A. et. al., 1989, Oncogene, 4:1299-1305; Lev, S., et. al., 1993, Mol. Cell. Biol., 13:2224-2234; Wennstrom, S., et. al., 1992, 267:13749-13756; Riedel, H., et. al., 1987, Science, 236:197-200; Seedorf, K., et. al.,1991, J. Biol. Chem., 266:12424-12431; Mares, J., et. al., 1992, Growth Factors, 6:93-101; Prigent, S. A. and Gullick, W. J., 1994, 13:2831-2841; Reich-Slotky, R., et. al., 1995, J. Biol. Chem., 270:29813-29818; Sistonen, L., et. al., 1989, J. Cell Biol., 109:1911-1919; Rizzo, C., et. al., 1996, J. Biol. Chem., 271:29497-29501; Riedel, H., 1994, J. Virol., 68:411-424; Sartor, C. I., et. al., 2001, Mol. Cell. Biol., 21:42654275; Chaika, O. V., et. al., 1997, J. Biol. Chem., 272:11968-11974; Piccinini, G., et. al., 2002, J. Biol. Chem., 277:2231-22239; Rizzo, C. et. al. 1996, J. Biol. Chem. 271:29497-29501).

In the practice of this invention, the hybrid receptor will preferably contain a transmembrane sequence fused between the ligand binding domain and the reporter polypeptide. Typical transmembrane domains contain about from 20 to 25 residues and show a hydropathy peak of about from 1.5 to 3.5. They contain a high proportion of residues having hydrophobic side chains, e.g. leucine, isoleucine, phenylalanine, valine and methionine. Suitable transmembrane sequences are obtained from the Ret receptor, in particular the human, rat or mouse Ret receptors, or from the transmembrane region ordinarily associated with the heterologous protein-tyrosine kinase receptor, or from integral membrane proteins of unrelated receptors, or may also be entirely synthetic.

In the practice of this invention, the hybrid receptor components can originate from any species whose genome encodes the appropriate receptor protein-tyrosine kinase component. The hybrid receptor components of this invention suitably originate from animals, including humans, other primates, rodents and insects, plants, fungi, microorganisms, parasites, and yeast, and any other suitable species. The species of origin for the Ret domain is preferably selected from human, mouse, rat, or primate, but can be from any other species possessing a Ret receptor. It is not necessary that the kinase polypeptide or transmembrane region be from the same species as the Ret domain.

The hybrid receptors of this invention are preferably synthesized in recombinant cell culture because they are generally too large and complex to be practically synthesized by in vitro methods that are available to the art today.

Thus, this invention provides a nucleic acid encoding a hybrid receptor comprising (a) a modified extracellular domain of the Ret receptor kinase, containing one or more amino acid residue substitutions, deletions or additions that render it capable of activating an intracellular receptor protein-tyrosine kinase domain in a ligand-independent manner, and (b) the kinase domain of a heterologous receptor protein-tyrosine kinase, said heterologous kinase domain being rendered in an active conformation by its association with the modified Ret extracellular domain. The nucleic acid can be a DNA or an RNA.

This invention also provides vectors comprising any such nucleic acids encoding the hybrid receptors of this invention, including vectors adapted for expression in a cell, which vector comprises the regulatory elements necessary for expression of the nucleic acid in the cell operatively linked to the nucleic acid encoding the receptor so as to permit expression thereof. Furthermore this invention also provides vectors which are plasmids.

This invention further provides host cells comprising any of the vectors described herein. The host cell is typically a eukaryotic cell, a mammalian cell, a human cell, an insect cell, a yeast cell or a prokaryotic cell, although is not limited to these. In one embodiment of this invention an NIH-3T3 cell is used.

Recombinant methods for synthesis of the hybrid receptors of this invention commence with the construction of a replicable vector containing nucleic acid that encodes the hybrid receptor. Vectors typically perform two functions in collaboration with compatible host cells. One function is to facilitate the cloning of the nucleic acid that encodes the hybrid receptor, i.e., to produce usable quantities of the nucleic acid. The other function is to direct the expression of the hybrid receptor. One or both of these functions are performed by the vector-host system. The vectors will contain different components depending upon the function they are to perform as well as the host cell that is selected.

This invention thus provides vectors that contain nucleic acid encoding the hybrid receptor. Typically, this will be DNA that encodes the hybrid receptor in its mature form linked at its amino terminus to a secretion signal. This secretion signal preferably is the signal presequence that normally directs the secretion of the wild-type Ret receptor to which the modified version of the Ret receptor extracellular domain is most closely related, or was derived. However, suitable secretion signals also include signals from other receptors or from secreted polypeptides of the same or related species.

The secreted hybrid receptor of this invention will lodge in the recombinant host membrane if it contains a transmembrane region. Ordinarily, hybrids are preferred that contain a transmembrane region that substantially retains structural fidelity, by virtue of the molecule's incorporation into the cell membrane. A preferred embodiment of this invention is the use of host cells expressing such a hybrid receptor for the identification or characterization of modulators of its protein-tyrosine kinase activity. On the other hand, if such a region is not present in the hybrid, then the hybrid may be secreted into the culture medium. The purification of transmembrane-deleted receptors is less complex than for membrane-bound receptors, because in the latter instance the hybrid receptor is more readily purified free of other cell membrane proteins. Thus in certain embodiments of this invention such receptors may be preferred. For example, in instances where a recombinant cell-bound hybrid receptor would exert an undesired biological effect on the host cell if induced to accumulate in high concentration in the cell membrane during the growth phase, such transmembrane-deleted receptors may be a useful alternative for assays of modulators of its protein-tyrosine kinase activity. Alternatively, this potential problem may be overcome by placing the nucleic acid encoding the hybrid receptor under the control of an inducible promoter. In embodiments of this invention where purification of the hybrid receptor is required, for example where the hybrid receptor is secreted into the cell medium, the hybrid receptor is readily purified by any of the protein purification techniques commonly practiced in the art, e.g. immunoaffinity chromatography. The recombinant hybrid receptor can also be engineered to contain a structural element or epitope to assist in its purification, e.g. poly-histidine, calmodulin-binding peptide, glutathione-S-transferase, or maltose-binding protein.

This invention also provides a membrane preparation isolated from any of the cells described above that contain vectors comprising nucleic acids that encode for and allow the expression of the recombinant hybrid receptor of this invention, wherein the membrane preparation comprises recombinantly produced hybrid receptor comprising (a) a modified extracellular domain of the Ret receptor kinase, containing one or more amino acid residue substitutions, deletions or additions that render it capable of activating an intracellular receptor protein-tyrosine kinase domain in a ligand-independent manner, and (b) the kinase domain of a heterologous receptor protein-tyrosine kinase, said heterologous kinase domain being rendered in an active conformation by its association with said Ret extracellular domain.

In the practice of this invention, for cloning vectors the hybrid receptor-encoding nucleic acid ordinarily is present together with a nucleic acid sequence that enables the vector to replicate in a selected host cell independent of the host chromosomes. This sequence is generally an origin of replication or an autonomously replicating sequence. Such sequences are well-known for a variety of bacteria, yeast and higher eukaryotic cells. The origin from the well-known plasmid pBR322 is suitable for E. coli bacteria, the 2.mu. plasmid origin for yeast and various viral origins for mammalian cells (SV40, polyoma, adenovirus or bovine papilloma virus). Less desirably, DNA is cloned by insertion into the genome of a host. This is readily accomplished with bacillus species, for example, by inserting into the vector DNMA that is complementary to bacillus genomic DNA. Transfection of bacillus with this vector results in homologous recombination with the genome and insertion of the hybrid receptor DNA. However, the recovery of genomic DNA encoding the hybrid receptor is more complex than obtaining exogenously replicated viral or plasmid DNA because restriction enzyme digestion is required to recover the hybrid receptor DNA from the genome of the cloning vehicle.

In the practice of this invention, expression and cloning vectors should contain a selection gene, also termed a selectable marker. This is a gene that encodes a protein necessary for the survival or growth of a host cell transformed with the vector. The presence of this gene ensures the growth of only those host cells that express the inserts. Typical selection genes encode proteins that (a) confer resistance to antibiotics or other toxins, e.g. ampicillin, neomycin, blasticidin, G-418, mycophenolic acid, hygromycin B, bleomycin, phleomycin, methotrexate or tetracycline, (b) complement auxotrophic deficiences, or (c) supply critical nutrients not available from complex media, e.g. the gene encoding D-alanine racemase for bacilli.

A suitable selection gene for use in yeast is the trp1 gene present in the yeast plasmid YRp7 (Stinchcomb et al., 1979, “Nature”, 282: 39; Kingsman et al., 1979, “Gene”, 7: 141; or Tschemper et al., 1980, “Gene”, 10: 157). The trp1 gene provides a selection marker for a mutant strain of yeast lacking the ability to grow in the absence of tryptophan, for example ATCC No. 44076 or PEP41 (Jones, 1977, “Genetics”, 85: 12). The presence of the trp1 lesion in the yeast host cell genome then provides an effective environment for detecting transformation by growth in the absence of tryptophan. Similarly, Leu2 deficient yeast strains (ATCC 20,622 or 38,626) are complemented by known plasmids bearing the Leu2 gene.

Examples of suitable selectable markers for mammalian cells are dihydrofolate reductase (DHFR), thymidine kinase or proteins for neomycin resistance. Such markers enable the identification of cells that were competent to take up the hybrid receptor nucleic acid. The mammalian cell transformants are placed under selection pressure, which only the transformants are uniquely adapted to survive by virtue of having taken up the marker. Selection pressure is imposed by culturing the transformants in successive rounds of cell culture, in which the concentration of selection agent in the medium is successively increased, thereby leading to amplification of both the selection gene and the DNA encoding the hybrid receptor. Increased quantities of hybrid receptor are synthesized from the amplified DNA.

For example, selection for DHFR transformed cells is conducted in a culture medium which lacks hypoxanthine, glycine, and thymidine. An appropriate host cell in this case is the Chinese hamster ovary (CHO) cell line deficient in DHFR activity, prepared and propagated as described by Urlaub and Chasin, 1980, “Proc. Nat'l. Acad, Sci. USA” 77: 4216.

A particularly useful DHFR is a mutant DHFR that is highly resistant to methotrexate (MTX) (EP 117,060A). This selection agent can be used with any otherwise suitable host, notwithstanding the presence of endogenous DHFR. One simply includes sufficient MTX in the medium to inactivate all of the endogenous DHFR, whereupon MTX selection becomes solely a function of amplification of the mutant DHFR DNA. Most eukaryotic cells which are capable of adsorbing MTX appear to be methotrexate sensitive. One such useful cell line is a CHO line, CHO-K1 (ATCC No. CCL 61).

Other methods, vectors and host cells suitable for adaptation to the synthesis of the hybrid receptor of this invention in recombinant vertebrate cell culture are described in M. J. Gething et al., Nature 293: 620-625 (1981); N. Mantei et al., Nature 281: 40-46; EP 117,060A; EP 117,058A; Molecular Cloning: a Laboratory Manual, 2001, 3^{rd} Edition, by Joseph Sambrook and Peter MacCallum, (the former Maniatis Cloning manual) (e.g. ISBN 0-87969-577-3); and Current Protocols in Molecular Biology, Ed. Fred M. Ausubel, et. al. John Wiley & Sons (e.g. ISBN 0-471-50338-X).

Expression vectors of this invention, unlike cloning vectors, should contain a promoter and/or other sequence that is recognized by the host organism for strong transcription of the hybrid receptor encoding DNA. This is generally a promoter homologous to the intended host. In the case of vectors for higher eukaryotes, enhancer sequences are useful for further increasing transcription from promoters. Unlike promoters, enhancers do not need to be located 5′ to the hybrid receptor encoding nucleic acid. Commonly used promoters for prokaryotes include the beta-lactamase and lactose promoter systems (Chang et al., 1978, “Nature”, 275: 615; and Goeddel et al., 1979, “Nature”, 281; 544), alkaline phosphatase, a tryptophan (trp) promoter system (Goeddel 1980, “Nucleic Acids Res.” 8: 4057 and EPO Appln. Publ. No. 36,776) and hybrid promoters such as the tac promoter (H. de Boer et al., 1983, “Proc. Nat'l. Acad. Sci. USA” 80: 21-25). However, other known microbial promoters are suitable. Their nucleotide sequences have been published, thereby enabling a skilled worker operably to ligate them to DNA encoding the hybrid receptor in plasmid vectors (Siebenlist et al., 1980, “Cell” 20: 269) using linkers or adaptors to supply any required restriction sites. Promoters for use in prokaryotic systems also will contain a Shine-Dalgarno (S.D.) sequence operably linked to the DNA encoding the hybrid receptor.

Suitable promoting sequences in yeast vectors for use in the practice of this invention include the promoters for metallothionein, 3-phosphoglycerate kinase (-Hitzeman et al., 1980, “J. Biol. Chem.”, 255: 2073) or other glycolytic enzymes (Hess et al., 1968, “J. Adv. Enzyme Reg.”, 7: 149; and Holland, 1978, “Biochemistry”, 17: 4900), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters for use in the practice of this invention, which have the additional advantage of transcription controlled by growth conditions, are the promoter regions for alcohol dehydrogenase 2, isocytochrome C, acid phosphatase, degradative enzymes associated with nitrogen metabolism, and the aforementioned metallothionein and glyceralidehyde-3-phosphate dehydrogenase, as well as enzymes responsible for maltose and galactose utilization. Suitable vectors and promoters for use in yeast expression are further described in R. Hitzeman et al., EP 73,657A.

In the practice of this invention, transcription from vectors in mammalian host cells is controlled by promoters and/or enhancers obtained from the genomes of bovine papilloma virus, vaccinia virus, polyoma virus, adenovirus 2, retroviruses, hepatitus-B virus, cytomegalovirus, spleen focus forming virus, murine stem cell virus, Moloney murine leukemia virus, and Simian Virus 40 (SV40), operably linked to the hybrid receptor nucleic acid. The early and late promoters of the SV40 virus are as conveniently obtained as an SV40 restriction fragment, which also contains the SV40 viral origin of replication (Fiers et al., 1978, “Nature”, 273: 113). Of course, promoters or enhancers from the host cell or related species also are useful herein. A suitable mammalian expression vector for practice of this invention is pcDNA3.1. Retrovirus vectors may also be used in the practice of this invention, including those with inducible elements, e.g. tetracycline responsive elements.

Nucleic acid of this invention is operably linked when it is placed into a functional relationship with another nucleic acid sequence. For example, DNA for a presequence or secretory leader is operably linked to DNA for a polypeptide if it is expressed as a preprotein which participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are contiguous and, in the case of a secretory leader, contiguous and in reading frame.

Expression vectors used in eukaryotic host cells of this invention (yeast, fungi, insect, plant, animal or human) will also contain sequences necessary for the termination of transcription and for stabilizing the mRNA. Such sequences are commonly available from the 3′-untranslated regions of eukaryotic or viral cDNAs. These regions contain regions that are transcribed as polyadenylated segments in the untranslated portion of the mRNA encoding the hybrid receptor. The 3′ untranslated regions also include transcription termination sites.

Suitable host cells for cloning or expressing the vectors herein are prokaryotes, yeast or higher eukaryotic cells. Prokaryotes include gram negative or gram positive organisms, for example E. coli or bacilli. A preferred cloning host is E. coli 294 (ATCC 31,446) although other gram negative or gram positive prokaryotes such as E. coli B, E. coli X1776 (ATCC 31,537), E. coli W3110 (ATCC 27,325), pseudomonas species, or Serratia Marcesans are suitable.

In addition to prokaryotes, eukaryotic microbes such as filamentous fungi or yeast are suitable hosts for the hybrid receptor encoding vectors. Saccharomyces cerevisiae, or common baker's yeast, is the most commonly used among lower eukaryotic host microorganisms. However, a number of other genera, species and strains are commonly available and useful herein.

The preferred host cells for the expression of functional hybrid receptors of this invention are cultures of cells derived from multicellular organisms. In many cases, hybrid receptors contain hydrophobic regions that are incompatible with lower microorganisms, require complex processing to properly form disulfide bonds and often require subunit processing. In addition, it is desirable to glycosylate the receptors in a fashion similar to the native receptors. All of these functions can be best performed by higher eukaryotic cells. In principle, any higher eukaryotic cell culture is workable, whether from vertebrate or invertebrate culture, although cells from mammals such as humans are preferred. Propagation of such cells in culture is per se well known. See Tissue Culture, Academic Press, Kruse and Patterson, editors (1973). Examples of useful mammalian host cell lines are VERO and HeLa cells, human 239 cells, quail QT6 cells, NIH-3T3 cells, Chinese hamster ovary cell lines, and WI38, BHK, COS-7 and MDCK cell lines.

Thus, this invention also provides a cell comprising a hybrid receptor, wherein the hybrid receptor comprises (a) a modified extracellular domain of the Ret receptor kinase, containing one or more amino acid residue substitutions, deletions or additions that render it capable of activating an intracellular receptor protein-tyrosine kinase domain in a ligand-independent manner, and (b) the kinase domain of a heterologous receptor protein-tyrosine kinase, said heterologous kinase domain being rendered in an active conformation by its association with said Ret extracellular domain. The cell of this invention can be eukaryotic, mammalian, human, insect or yeast. The cell comprising the hybrid receptor of this invention can be a stable or transient transfectant.

This invention further provides a method for producing a hybrid receptor comprising (a) a modified extracellular domain of the Ret receptor kinase, containing one or more amino acid residue substitutions, deletions or additions that render it capable of activating an intracellular receptor protein-tyrosine kinase domain in a ligand-independent manner, and (b) the kinase domain of a heterologous receptor protein-tyrosine kinase, said heterologous kinase domain being rendered in an active conformation by its association with said Ret extracellular domain, said method comprising growing a host cell comprising a vector as described above that is adapted for expression in the cell, said vector comprising the regulatory elements necessary for expression of the hybrid receptor encoding nucleic acid in the cell operatively linked to the nucleic acid encoding the hybrid receptor so as to permit expression thereof under suitable conditions permitting production of said hybrid receptor, and recovering the hybrid receptor. In one embodiment of this method, the method further comprises preparing from the recovered hybrid receptor, a membrane preparation containing the hybrid receptor. In an alternative embodiment of this method, the method further comprises purifying the recovered hybrid receptor.

The hybrid receptors of this invention are employed in drug screening assays by a process that fundamentally comprises incubating the hybrid receptor with the test sample, controls and (optionally) standards, followed by measuring a change in the activity of the heterologous kinase domain of the hybrid receptor. Since binding of a modulator to the hybrid receptor kinase domain causes a change in the kinase activity it is within the scope hereof to detect such change by any one of several methods. Typically, one measures changes in the protein binding or enzymatic activity of the hybrid receptor. In one embodiment an antibody specific for the activated conformation or autophosphorylated domain is utilized, and the binding of this antibody to the hybrid receptor is measured after the receptor has been incubated with the candidate drug. This assay is conducted in the same fashion as conventional immunoassay methods for any protein antigen, e.g. using ELISA or immunoblotting (Western blotting) methods (e.g. see Using Antibodies, A Laboratory Manual, edited by Harlow, E. and Lane, D., 1999, Cold Spring Harbor Laboratory Press, (e.g. ISBN 0-87969-544-7)). Antibodies are known in the art that are capable of binding phosphotyrosine-containing proteins and are suitable for use in many different assay formats (e.g. Wang, 1985, “Mol. and Cell. Biol” 5(12): 3640-3643; Ross et al., 1981, “Nature” 294: 654; and Pang et al., 1985, “Arch. Biochem. Biophys.” 242(1): 176; Stewart, A. A., in Protein Phosphorylation, A Practical Approach, 1993, Ed. Hardie, D. G., p145-171,). Antibodies are also known that bind to specific phosphopeptides, or that bind to specific active kinase conformations, and would be suitable for use in the practice of this invention.

This invention thus provides a composition comprising a hybrid receptor protein-tyrosine kinase selected from: (i) a cell comprising a hybrid receptor, wherein the hybrid receptor comprises (a) a modified extracellular domain of the Ret receptor kinase, containing one or more amino acid residue substitutions, deletions or additions that render it capable of activating an intracellular receptor kinase domain in a ligand-independent manner, and (b) the kinase domain of a heterologous receptor protein-tyrosine kinase, said kinase domain being rendered in an active conformation by its association with said Ret extracellular domain, (ii) a membrane preparation isolated from a cell comprising a hybrid receptor, wherein the hybrid receptor comprises (a) a modified extracellular domain of the Ret receptor kinase, containing one or more amino acid residue substitutions, deletions or additions that render it capable of activating an intracellular receptor protein-tyrosine kinase domain in a ligand-independent manner, and (b) the kinase domain of a heterologous receptor protein-tyrosine kinase, said heterologous kinase domain being rendered in an active conformation by its association with said Ret extracellular domain, or (iii) a hybrid receptor comprising (a) a modified extracellular domain of the Ret receptor kinase, containing one or more amino acid residue substitutions, deletions or additions that render it capable of activating an intracellular receptor protein-tyrosine kinase domain in a ligand-independent manner, and (b) the kinase domain of a heterologous receptor protein-tyrosine kinase, said heterologous kinase domain being rendered in an active conformation by its association with said Ret extracellular domain, for use in a method to detect a modulator of a receptor protein-tyrosine kinase.

Furthermore, this invention provides a method for detecting a modulator of a selected receptor protein-tyrosine kinase, comprising, (a) providing a hybrid receptor comprising a modified extracellular domain of the Ret receptor kinase, containing one or more amino acid residue substitutions, deletions or additions that render it capable of activating an intracellular receptor protein-tyrosine kinase domain in a ligand-independent manner, and the heterologous kinase domain of the selected receptor protein-tyrosine kinase; (b) incubating the hybrid receptor with a test sample suspected to contain a modulator of the receptor protein-tyrosine kinase activity; (c) detecting a change in activity of the receptor protein-tyrosine kinase; and (d) correlating said change with the presence of the modulator in the test sample.

This invention also provides a method for detecting a modulator of a selected receptor protein-tyrosine kinase, comprising (a) providing a cell comprising a hybrid receptor, wherein the hybrid receptor comprises a modified extracellular domain of the Ret receptor kinase, containing one or more amino acid residue substitutions, deletions or additions that render it capable of activating an intracellular receptor protein-tyrosine kinase domain in a ligand-independent manner, and the heterologous kinase domain of the selected receptor protein-tyrosine kinase; (b) incubating the cell with a test sample suspected to contain a modulator of the receptor protein-tyrosine kinase activity; (c) detecting a change in activity of the receptor protein-tyrosine kinase; and (d) correlating said change with the presence of the modulator in the test sample.

This invention also provides a method for detecting a modulator of a selected receptor protein-tyrosine kinase, comprising (a) providing a membrane preparation comprising a hybrid receptor, wherein the hybrid receptor comprises a modified extracellular domain of the Ret receptor kinase, containing one or more amino acid residue substitutions, deletions or additions that render it capable of activating an intracellular receptor protein-tyrosine kinase domain in a ligand-independent manner, and the heterologous kinase domain of the selected receptor protein-tyrosine kinase; (b) incubating the membrane preparation with a test sample suspected to contain a modulator of the receptor protein-tyrosine kinase activity; (c) detecting a change in activity of the receptor protein-tyrosine kinase; and (d) correlating said change with the presence of the modulator in the test sample.

In the practice of this invention the modulator detected by a change in activity of the hybrid receptor protein-tyrosine kinase of this invention can be an inhibitor or an activator of the kinase activity.

This invention also provides a process for preparing a composition, for example, a pharmaceutical composition which comprises admixing a carrier, for example, a pharmaceutically acceptable carrier, and a pharmaceutically effective amount of a chemical compound identified by a process in accordance with this invention or a novel structural and functional analog or homolog thereof.

This invention provides a method of screening a plurality of chemical compounds not known to modulate the heterologous protein-tyrosine kinase activity of the hybrid receptor of this invention, to identify a compound which modulates the activity of the heterologous protein-tyrosine kinase, which comprises: (a) contacting cells transfected with and expressing hybrid receptor of this invention with the plurality of compounds not known to modulate the heterologous protein-tyrosine kinase activity, under conditions permitting modulation of the heterologous protein-tyrosine kinase activity; (b) determining whether the activity of the heterologous protein-tyrosine kinase is changed in the presence of one or more of the compounds; and if so (c) separately determining whether the activity of the heterologous protein-tyrosine kinase is modulated by any compound included in the plurality of compounds, so as to thereby identify each compound which modulates the activity of the heterologous protein-tyrosine kinase.

It is contemplated that this hybrid receptor of this invention will serve as a valuable tool for designing drugs for treating various pathophysiological conditions such as chronic and acute inflammation, arthritis, autoimmune diseases, transplant rejection, graft versus host disease, bacterial, fungal, protozoan and viral infections, septicemia, AIDS, pain, psychotic and neurological disorders, including anxiety, depression, schizophrenia, dementia, mental retardation, memory loss, epilepsy, neurological disorders, neuromotor disorders, respiratory disorders, asthma, eating/body weight disorders including obesity, bulimia, diabetes, anorexia, nausea, hypertension, hypotension, vascular and cardiovascular disorders, ischemia, stroke, cancers, ulcers, urinary retention, sexual/reproductive disorders, circadian rhythm disorders, renal disorders, bone diseases including osteoporosis, benign prostatic hypertrophy, gastrointestinal disorders, nasal congestion, dermatological disorders such as psoriasis, allergies, Parkinson's disease, Alzheimer's disease, acute heart failure, angina disorders, delirium, dyskinesias such as Huntington's disease or Gille's de la Tourette's syndrome, among others. The hybrid receptor may also serve as a valuable tool for designing drugs for chemoprevention.

Membrane preparations comprising the hybrid receptor of this invention are derived from cells comprising a hybrid receptor. An additional embodiment of this invention includes preparations of the hybrid receptor of this invention prepared by detergent solubilization of such membrane preparations, typically achieved by the addition to the membranes of one or more non-ionic detergents.

In the practice of this invention, detection of a change in activity of the hybrid receptor protein kinase may be achieved by immunoassay of changes in the hybrid receptor kinase activity, using polyclonal or monoclonal antibodies. Immunoreactive fragments of these antibodies or a cocktail of antibodies can also be used to practice the invention. These antibodies can be labeled directly with a reporter or indirectly with a member of a specific binding pair using conventional techniques.

In the practice of this invention any of the commonly used immunoassay techniques may be used for isolation of hybrid receptor protein, or quantitation of the activity of hybrid receptor protein, including immunoprecipitation, immunoblotting (Western blotting), and ELISA assays. In one preferred embodiment, an anti-Ret antibody is used for isolation of the hybrid receptor protein, for example by immunoprecipitation, and change in activity of the hybrid receptor protein is quantitated using a labeled antiphosphotyrosine antibody to assess autophosphorylation of the hybrid receptor, or by its activity on a peptide or protein substrate. In a further preferred embodiment, an ELISA assay is used in which the hybrid receptor protein is initially captured using an anti-Ret antibody, and autophosphorylation then assessed in a second step using a labeled antiphosphotyrosine antibody.

For ELISA assays, specific binding pairs can be of the immune or non-immune type. Immune specific binding pairs are exemplified by antigen-antibody systems or hapten/anti-hapten systems. There can be mentioned fluorescein/anti-fluorescein, dinitrophenyl/anti-dinitrophenyl, biotin/anti-biotin, peptide/anti-peptide and the like. The antibody member of the specific binding pair can be produced by customary methods familiar to those skilled in the art. Such methods involve immunizing an animal with the antigen member of the specific binding pair. If the antigen member of the specific binding pair is not immunogenic, e.g., a hapten, it can be covalently coupled to a carrier protein to render it immunogenic.

Non-immune binding pairs include systems wherein the two components share a natural affinity for each other but are not antibodies. Exemplary non-immune pairs are biotin-streptavidin, intrinsic factor-vitamin B₁₂, folic acid-folate binding protein and the like.

A variety of methods are available to covalently label antibodies with members of specific binding pairs. Methods are selected based upon the nature of the member of the specific binding pair, the type of linkage desired, and the tolerance of the antibody to various conjugation chemistries. Biotin can be covalently coupled to antibodies by utilizing commercially available active derivatives. Some of these are biotin-N-hydroxy-succinimide which binds to amine groups on proteins; biotin hydrazide which binds to carbohydrate moieties, aldehydes and carboxyl groups via a carbodiimide coupling; and biotin maleimide and iodoacetyl biotin which bind to sulfhydryl groups. Fluorescein can be coupled to protein amine groups using fluorescein isothiocyanate. Dinitrophenyl groups can be coupled to protein amine groups using 2,4-dinitrobenzene sulfate or 2,4-dinitrofluorobenzene. Other standard methods of conjugation can be employed to couple monoclonal antibodies to a member of a specific binding pair including dialdehyde, carbodiimide coupling, homofunctional crosslinking, and heterobifunctional crosslinking. Carbodiimide coupling is an effective method of coupling carboxyl groups on one substance to amine groups on another. Carbodiimide coupling is facilitated by using the commercially available reagent 1-ethyl-3-(dimethyl-aminopropyl)-carbodiimide (EDAC).

Homobifunctional crosslinkers, including the bifunctional imidoesters and bifunctional N-hydroxysuccinimide esters, are commercially available and are employed for coupling amine groups on one substance to amine groups on another. Heterobifunctional crosslinkers are reagents which possess different functional groups. The most common commercially available heterobifunctional crosslinkers have an amine reactive N-hydroxysuccinimide ester as one functional group, and a sulfhydryl reactive group as the second functional group. The most common sulfhydryl reactive groups are maleimides, pyridyl disulfides and active halogens. One of the functional groups can be a photoactive aryl nitrene, which upon irradiation reacts with a variety of groups.

The detectably-labeled antibody or detectably-labeled member of the specific binding pair is prepared by coupling to a reporter, which can be a radioactive isotope, enzyme, fluorogenic, chemiluminescent or electrochemical materials. Two commonly used radioactive isotopes are ¹²⁵I and ³H. Standard radioactive isotopic labeling procedures include the chloramine T, lactoperoxidase and Bolton-Hunter methods for ¹²⁵I and reductive methylation for ³H. The term “detectably-labeled” refers to a molecule labeled in such a way that it can be readily detected by the intrinsic enzymic activity of the label or by the binding to the label of another component, which can itself be readily detected.

Enzymes suitable for use in this invention include, but are not limited to, horseradish peroxidase, alkaline phosphatase, β-galactosidase, glucose oxidase, luciferases, including firefly and renilla, β-lactamase, urease, green fluorescent protein (GFP) and lysozyme. Enzyme labeling is facilitated by using dialdehyde, carbodiimide coupling, homobifunctional crosslinkers and heterobifunctional crosslinkers as described above for coupling an antibody with a member of a specific binding pair.

The labeling method chosen depends on the functional groups available on the enzyme and the material to be labeled, and the tolerance of both to the conjugation conditions. The labeling method used in the present invention can be one of, but not limited to, any conventional methods currently employed including those described by Engvall and Pearlmann, Immunochemistry 8, 871 (1971), Avrameas and Ternynck, Immunochemistry 8, 1175 (1975), Ishikawa et al., J. Immunoassay 4(3):209-327 (1983) and Jablonski, Anal. Biochem. 148:199 (1985).

Labeling can be accomplished by indirect methods such as using spacers or other members of specific binding pairs. An example of this is the detection of a biotinylated antibody with unlabeled streptavidin and biotinylated enzyme, with streptavidin and biotinylated enzyme being added either sequentially or simultaneously. Thus, according to the present invention, the antibody used to detect can be detectably-labeled directly with a reporter or indirectly with a first member of a specific binding pair. When the antibody is coupled to a first member of a specific binding pair, then detection is effected by reacting the antibody-first member of a specific binding complex with the second member of the binding pair that is labeled or unlabeled as mentioned above.

Moreover, the unlabeled detector antibody can be detected by reacting the unlabeled antibody with a labeled antibody specific for the unlabeled antibody. In this instance “detectably-labeled” as used above is taken to mean containing an epitope by which an antibody specific for the unlabeled antibody can bind. Such an anti-antibody can be labeled directly or indirectly using any of the approaches discussed above. For example, the anti-antibody can be coupled to biotin which is detected by reacting with the streptavidin-horseradish peroxidase system discussed above.

In one embodiment of this invention biotin is utilized. The biotinylated antibody is in turn reacted with streptavidin-horseradish peroxidase complex. Orthophenylenediamine, 4-chloro-naphthol, tetramethylbenzidine (TMB), ABTS, BTS or ASA can be used to effect chromogenic detection.

In one preferred immunoassay format for practicing this invention, a forward sandwich assay is used in which the capture reagent (e.g. anti-Ret antibodies) has been immobilized, using conventional techniques, on the surface of a support. Suitable supports used in assays include synthetic polymer supports, such as polypropylene, polystyrene, substituted polystyrene, e.g. aminated or carboxylated polystyrene, polyacrylamides, polyamides, polyvinylchloride, glass beads, agarose, or nitrocellulose.

In the practice of this invention, determination of hybrid receptor kinase activity may also be achieved by methods that directly or indirectly measure the binding to the hybrid receptor of a non-immune binding protein with which it normally interacts, e.g. an SH-2 domain binding protein. The association of the binding protein is monitored in a similar fashion as antibody binding.

Further, in the practice of this invention, an alternative detection method for activity changes in the hybrid receptor of this invention, particularly when assay of purified hybrid receptor proteins, or a hybrid receptor protein in a membrane preparation is contemplated, is an assay for protein or peptide phosphotransferase activity, whereby activity is monitored by the incorporation of radiophosphorus into the hybrid receptor through autophosphorylation with p³² phosphate, or by incorporation of radiophosphorus into an alternative substrate protein or peptide.

Further, in the practice of this invention, it is within the scope herein to measure changes in the activity of hybrid receptors by methods other than enzymological activity or polypeptide interactions, particularly when assay of purified hybrid receptor proteins is contemplated. One such method comprises binding an organic moiety to the hybrid receptor that undergoes a change in character upon binding a modulator compound. For example, the kinase domain is labeled with a stable free radical, a chemiluminescent group or a fluorescent molecule such as fluorescein isothiocyanate. Each of these labels are well known in the diagnostic immunochemistry art and conventional methods are well known for covalently linking them to proteins. These methods are useful for labeling the hybrid receptor in the same fashion as other proteins. Changes in the conformation of the receptor polypeptide upon the binding of a candidate drug to the kinase domain are detected by changes in the label. For example, the rotational moment of a stable free radical label will be increased or decreased by changes in polypeptide conformation. Similarly, the fluorescence or luminescence of reporter polypeptide labels will change upon the binding of modulator or drug candidate to the receptor because of the reorientation of polypeptide species that engage in intramolecular energy transfers. This is detected by changes in the intensity, polarization or wavelength of the label molecule; typically, one detects the enhancement or quenching of the label fluorescence or chemiluminescence. The advantage of this labeled receptor method is that the candidate drug assay is conducted exclusively in aqueous solution and no phase separation is required. This permits ready automation of the screening method.

This invention also provides cellular assays where rather than directly monitoring the kinase activity of the hybrid receptor of the invention, a downstream signal transduction event or activity is assayed (e.g. PI-3 kinase, AKT/protein kinase B), or a transcriptional activation event is monitored. The latter is readily assayed by including in the cell a promoter-reporter construct that is responsive to activation of the signal transduction pathway activated by the hybrid receptor of the invention. Many suitable reporters are well known in the art, e.g. firefly luciferase.

Many alternative experimental methods known in the art may be successfully substituted for those specifically described herein in the practice of this invention, as for example described in many of the excellent manuals and textbooks available in the areas of technology relevant to this invention (e.g. Using Antibodies, A Laboratory Manual, edited by Harlow, E. and Lane, D., 1999, Cold Spring Harbor Laboratory Press, (e.g. ISBN 0-87969-544-7); Roe B. A. et. al. 1996, DNA Isolation and Sequencing (Essential Techniques Series), John Wiley & Sons.(e.g. ISBN 0-471-97324-0); Methods in Enzymology: Chimeric Genes and Proteins”, 2000, ed. J. Abelson, M. Simon, S. Emr, J. Thorner. Academic Press; Molecular Cloning: a Laboratory Manual, 2001, 3^{rd} Edition, by Joseph Sambrook and Peter MacCallum, (the former Maniatis Cloning manual) (e.g. ISBN 0-87969-577-3); Current Protocols in Molecular Biology, Ed. Fred M. Ausubel, et. al. John Wiley & Sons (e.g. ISBN 0-471-50338-X); Current Protocols in Protein Science, Ed. John E. Coligan, John Wiley & Sons (e.g. ISBN 0-471-11184-8); and Methods in Enzymology: Guide to protein Purification, 1990, Vol. 182, Ed. Deutscher, M. P., Acedemic Press, Inc. (e.g. ISBN 0-12-213585-7)), or as described in the many university and commercial websites devoted to describing experimental methods in molecular biology.

This invention will be better understood from the Experimental Details which follow. However, one skilled in the art will readily appreciate that the specific methods and results discussed are merely illustrative of the invention as described more fully in the claims which follow thereafter, and are not to be considered in any way limited thereto.

### Experimental Details

### Materials and Methods

Cell Lines and Reagents

NIH 3T3 cells were purchased from the American Type Culture Collection and maintained in Dullbecco's modified Eagle's medium (DMEM) (Invitrogen) supplemented with 10% Fetal Bovine Serum (FBS). pcDNA 3.1 mammalian expression vector, PCR Blunt cloning vector, and DH5α competent E. coli cells were purchased from Invitrogen Life Corporation. PCR reagents were from Roche Molecular Systems, Inc. (N808-0228). Anti-Ret antibodies were from R & D systems, Inc (MAB718) and Santa Cruz Biotechnology, Inc. (sc-13140). Anti phosphotyrosine-HRP antibodies were from Calbiochem (525320). Restriction endonucleases were from New England Biolabs, Inc. The primers used for PCR were synthesized by ACGT, Inc. Poly (Glu,Tyr) 4:1 was from Sigma (P-0275). Horseradish peroxidase (HRP) substrate, ABTS [(2′2-azino-di(3-ethybenzthiazoline-6sulfonate)] was from Kirkegaard & Perry Labs, Inc., and Super Signal ELISA Femto Maximum Sensitivity Substrate was from Pierce (37075). Regular Western Blotting Detection Reagents were from Amersham Biosciences (RPN2106). Transfection reagents were purchased from Roche.

Construction and Expression of Ret^{C634W}/Tie2 in 3T3 Cells

The cDNA encoding for human Tie2 intracellular domain (Tie2^{WT}, 770-1123) or a c-terminal 16 amino acid deletion form of human Tie2 intracellular domain (Tie^{ΔC}, 770-1107) was generated by PCR using cDNA containing full-length human Tie2 as the template. The following synthesized oligonucleotides were employed as primer pairs for amplification of Tie2^{WT} and Tie2^{ΔC}. Primer pair for PCR Tie2^{WT} is 5′-CCTAGGATCCAAGAGGGCAAATGTGCAAAG-3′ (SEQ I.D. NO:1) and 5-GAAAGGGAAACAGAGGGAATTCAGATGTTC -3′ (SEQ I.D. NO:2), while the primer pair for PCR Tie2^{ΔC} is 5′-CCTAGGATCCAAGAGGGCAAATGTGCAAAG -3′ (SEQ I.D. NO:3) and 5′-CCTGCATAAGTAAACTTCTCAATAAAGCGTGGT ATTC-3′ (SEQ I.D. NO:4). In both cases, the 5′ primer contains an engineered BamH1 site, and 3′ primers contain an engineered EcoR1 site. For Tie2^{ΔC}, an engineered stop codon, TAA was also introduced prior to the EcoR1 site. The PCR reactions were conducted under the following sequential conditions: 1 cycle of 94° C. for 2 minutes, 25 cycles of denature (94° C. for 10 seconds), annealing (60° C. for 30 seconds) and elongation (72° C. for 1 minutes), as well as 1 cycle of 72° C. for 7 minutes. The resultant cDNAs were sequenced (ACGT, Inc.), and subsequently digested with BamH1 and EcoR1. The BamH1-EcoR1 fragments were then cloned between BamH1 and EcoR1 sites of pcDNA3.1 mammalian expression vector. Colonies were grown up and screened for clones having the insertion of Tie2 kinase domain cDNA fragments by digesting with BamH1 and EcoR1. The resulting plasmid was designated pTie2^{WT} or pTie2^{ΔC}.

The cDNA of human Ret extracellular domain having a mutation of cysteine 634 to tryptophan (encoding the amino acid sequence 1-656) was amplified by PCR with two synthesized oligonucleotides (by ACGT, Inc.). The 5′ primer sequence is 5′-TATAGATCTTGGCCCCAGCGCGCACGGGCGATGGCGAA-3′ (SEQ I.D. NO:5), and 3′primer sequence is 5′-TATAGATCTGATGCAGAAGGCAACAG CAG-3′ (SEQ I.D. NO:6). They both contain an engineered Bgl II site. 50 pmol of each primer per reaction was used. The template of the PCR reaction was a full-length human Ret cDNA that contains the mutation of cysteine 634 to tryptophan (10 ng). The PCR was started with a denature at 94° C. for 2 minutes (1 cycle) followed by 25 cycles of denature (94° C. for 10 seconds), annealing (60° C. for 30 seconds), and elongation (72° C. for 2 minutes). Before the reaction was stopped, an elongation of 7 minutes at 72° C. was performed. The PCR product of human Ret extracellular domain cDNA was subsequently cloned into PCR blunt vector (Invitrogen). The positive clone containing the Bgl II fragment of Ret extracellular domain with the cysteine mutation was confirmed by DNA sequencing (ATCG, Inc.). The Bgl II fragment was then isolated by gel extraction (Qiagen QIAquick Gel Extraction Kit) according to the manufacturer's instruction, and subsequently inserted into the dephosphorylated BamH1 site of pTie2^{WT} or pTie2^{ΔC}. The dephosphorylation of BamH1 cohesive termini was carried out by alkaline phosphatase (New England Biolabs). As BamH1 and Bgl II are comparable sites, the insertion of Bgl II fragments generated two orientations. The orientation necessary for expression of the chimera mRNA was identified by digesting with Kpn1. This resulting expression plasmid was designated pRet^{C634W}/Tie2^{WT} or pRet^{C634W}/Tie2^{ΔC}.

NIH 3T3 cells were seeded at 5×10⁵ cells/well in 2 ml DMEM supplemented with 10% FBS per well in 6-well plates. On Day 2, the growth medium was replaced with fresh medium, and pRet^{C634W}/Tie2^{WT} or pRet^{C634W}/Tie2^{ΔC} was introduced into 3T3 cells by FuGene-6 transfection reagent following the manufacturer's instruction (Roche). After 48 hours of transfection, the cells were either used directly for conducting experiments or for generating stable cell lines expressing Ret^{C634W}/Tie²^{WT} or Ret^{C634W}/Tie2^{ΔC} chimeric receptor by selecting the cells with neomycin at the concentration of 800 ug/mL. The neomycin resistant colonies were expanded, and expression of the chimeric receptors were analyzed by Western blotting with anti-Ret antibody (Santa Cruz, sc-13140).

Ligand Independent Autophosphorylation of Chimeric Receptors

pRet^{C634W}/Tie2^{WT}, pRet^{C634W}/Tie2^{ΔC} or mock transfected NIH 3T3 cells grown for 48 hours, were pre-treated with or without 1 mM Na₃VO₄ for the indicated period. Then, the cells were washed with cold PBS twice before they were lysed on ice in 0.5 ml cold TGH buffer (1% Triton-100, 10% glycerol, 50 mM Hepes [pH 7.4]) supplemented with 150 mM NaCl, 1.5 mM MgCl₂, 1 mM EDTA and fresh protease and phosphatase inhibitors (10 μg/ml leupeptin, 25 ug/ml aprotinin, 50 μg/ml phenylmethylsulfonyl fluoride [PMSF] and 200 μM Na₃VO₄), as described by Ji et al., 1999, “Mol.Cell. Biol.” 19:4961-4970. Cell lysates were centrifuged at 14,000 RPM to pellet cellular debris, transferred to a new tube containing 2 μg anti-Ret (R & D systems, MAB718) pre-coupled to Protein G agarose (Sigma), and incubated with agitation for 2 hours at 4° C. The Protein G captured antibody-protein complexes were washed three times with cold TGH buffer. The samples were boiled, and the immunoprecipitated chimeric receptors were separated on a 4-12% gradient SDS polyacrylamide gel. Following transfer to nitrocellulose membranes, the proteins were probed with anti-phosphotyrosine-HRP. The bound antibody was detected by enhanced chemiluminescence (ECL).

In Vitro Kinase Assay

Under the same conditions described above, the immunocomplexes derived from the 3T3 cells expressing Ret^{C634W}/Tie2^{WT} or Ret^{C634W}/Tie2^{ΔC} chimera were directly used in the in vitro kinase assay. An equal amount of Protein G captured chimeric receptor was added to an Immulon-4 96-well plate (Thermo Labsystems) coated with 2 μg/well of substrate poly-glu-tyr (4:1 ratio) in phosphorylation buffer (50 mM Hepes, pH 7.4, 125 mM NaCl, 24 mM MgCl₂, 1 mM MnCl₂, 1% glycerol, 200 μM Na₃VO₄, 2 mM DTT). The enzymatic reaction was initiated by addition of ATP at a final concentration of 25 μM. After incubation at room temperature for indicated period, the plates were washed with 2 mM imidazole buffered saline with 0.02% Tween-20. Then the plate was incubated with 18.75 ng/well of anti-phosphotyrosine-HRP antibody (Calbiochem) diluted in PBS containing 3% BSA, 0.5% Tween-20 and 200 μM Na₃VO₄ for 2 hours at room temperature. Following 3×250 μl washes, the bound anti-phosphotyrosine-HRP was detected by incubation with 100 μl/well ABTS (Kirkegaard & Perry Labs, Inc.) for 30 minutes at room temperature. The reaction was stopped by the addition of 100 μl/well 1% SDS, and the phosphotyrosine dependent signal was measured by a plate reader at 405/490 nm.

Cell-Based Autophosphorylation Assay

NIH 3T3 cells stably expressing Ret^{C634W}/Tie2^{ΔC} were seeded at 1×10⁴ cells/well in 0.1 ml DMEM supplemented with 10% FBS per well in 96-well plates. On Day 2, a compound was diluted in 100% DMSO, added to the cells at six final concentrations in duplicates (20, 6.6, 2.2, 0.74, 0.25 and 0.082 μM), and incubated at 37° C. for 2 hours. The media was then removed and the cells were washed once with PBS, then lysed with cold TGH buffer (1% Triton-100, 10% glycerol, 50 mM Hepes [pH 7.4]) supplemented with 150 mM NaCl, 1.5 mM MgCl, 1 mM EDTA and fresh protease and phosphatase inhibitors (10 μg/ml leupeptin, 25 μg/ml aprotinin, 50 μg/ml phenylmethylsulfonly fluoride [PMSF] and 200 μM Na₃VO₄). Cell lysates were transferred to a 96-well microlite2 plate (Dynex #7417) coated with 10 ng/well of anti-Ret antibody (R & D Systems, MAB718), and incubated at 4° C. overnight. Following washing with TGH buffer, the plate was incubated with anti-phosphotyrosine-HRP for 2 hours at room temperature. The autophosphotyrosine was then detected by addition of Super Signal ELISA Femto Maximum Sensitivity Substrate (Pierce) and chemiluminescence was read on a Wallac Victor² 1420 Multilabel Counter. The IC50 curves of the compounds were plotted using a ExcellFit program. For NIH 3T3 cells stably expressing Ret^{C634W}/Tie2^{WT} or a situation where a higher signal would be desired, the cells were pre-treated with 1 mM Na₃VO₄ for 10 minutes at room temperature prior to being lysed.

Membrane Preparations

Cell membranes expressing the hybrid receptor protein according to this invention are useful for certain types of assays, including autophosphorylation assays, peptide phosphorylation assays, and others. The specifics of preparing such cell membranes may in some cases be determined by the nature of the ensuing assay or cell type, but typically involve harvesting whole cells and disrupting the cell pellet by sonication in ice cold buffer (e.g. 20 mM Tris-HCl, 5 mM EDTA, pH 7.4). The resulting crude cell lysate is cleared of cell debris by low speed centrifugation at 200 g for 5 min at 4° C. The cleared supernatant is then centrifuged at 40,000 g for 20 min at 4° C., and the resulting membrane pellet is washed by suspending in ice cold buffer and repeating the high speed centrifugation step. The final washed membrane pellet is resuspended in assay buffer. Protein concentrations are determined by the method of Bradford (Bradford, M. M., 1976, Anal. Biochem. 72: 248-254) using bovine serum albumin as a standard. The membranes may be used immediately or frozen for later use.

Results

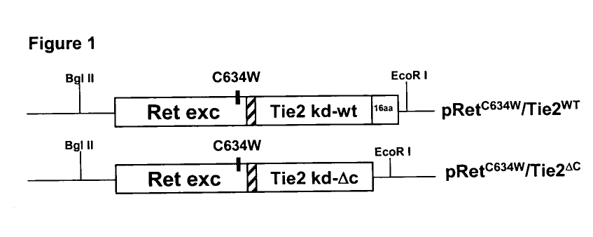

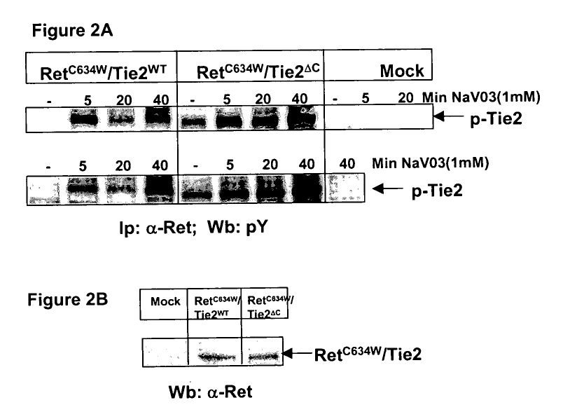

In order to test if the point mutation of cysteine 634 in the Ret extracellular domain (Ret^{C634W}) would result in the ligand-independent kinase activation of a heterologous receptor, a eukaryotic expression vector encoding a chimeric receptor was engineered (FIG. 1). This receptor consists of the Ret^{C634W} fused at its C-termini to the N-termini of either full-length intracellular domain of Tie2 receptor tyrosine kinase or a C-terminal 16 amino acid deletion form of intracellular domain of the Tie2 receptor tyrosine kinase. This hybrid receptor was designed Ret^{C634W}/Tie2^{WT} or Ret^{C634W}/Tie2^{ΔC}. NIH 3T3 cells were transiently transfected with pRet^{C634W}/Tie2^{WT} or pRet^{C634W}/Tie₂^{ΔC}. The expression levels of these two chimeras are similar, determined by Western blotting with anti-Ret antibody (Santa Cruz, sc-13104) (FIG. 2B). Lysates prepared from these transfectants were immunoprecipitated with an anti-Ret antibody (R & D systems, MAB718), which recognizes only the extracellular domain of the Ret receptor. The resulting immunocomplexes were analysed by SDS-polyacrylamide gel electrophoresis, and autophosphorylation of the Tie2 was analyzed by Western blotting with anti-phosphotyrosine-HRP. As shown in FIG. 2A, a steady-state phosphotyrosine content was observed in the cells expressing Ret^{C634W}/Tie2^{ΔC}. However, no significant phosphotyrosine was revealed with respect to the wildtype Tie2 fused to the Ret^{C634W}. These results were consistent with recent structure and biochemical studies that indicated a role of Tie2 C-terminus in the negative regulatory of the Tie2 kinase activity (Shewchuk, et al., Structure 2000; 8:1105-1113, Niu et al., JBC 2002; 277:31768-31773). To test whether the wildtype Tie2 kinase domain in Ret^{C634W}/Tie2^{WT} chimera could be activated, NIH 3T3 cells expressing Ret^{C634W}/Tie2^{WT} were treated with 1 mM Na₃VO₄ prior to immunoprecipitation and phosphotyrosine detection. FIG. 2A shows that full-length Tie2 kinase was significantly activated when the cells were treated with Na₃VO₄. Moreover, the activity of the C-terminal deletion form of the Tie2 kinase was also enhanced under the same conditions, indicating an additional negative regulation, likely through protein tyrosine phosphatases. Taken together, these data indicated that the point mutation of cysteine 634 to tryptophan in the Ret extracellular domain also results in a ligand-independent activation of Tie2 kinase under physiological conditions.

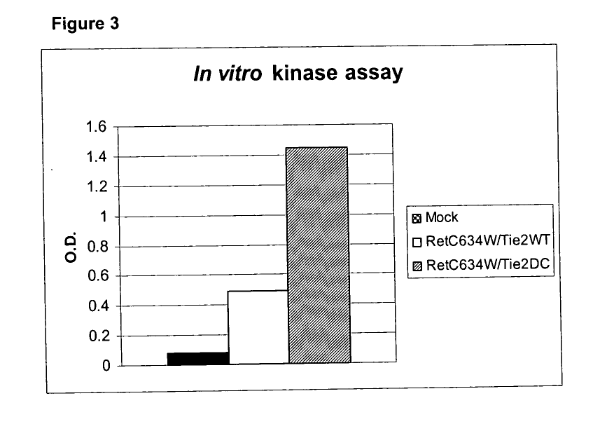

To ascertain that Tie2 kinase was constitutively active by Ret^{C634W} mutant, we next examined the tyrosine phosphorylation of an exogenous substrate by performing an in vitro kinase assay. In the presence of 25 μM ATP, an equal amount of the immunocomplexes derived directly from 3T3 cells expressing Ret^{C634W}/Tie2^{WT} or Ret^{C634W}/Tie2^{ΔC} chimera, as described above was added to a 96-well plate that was pre-coated with the substrate, polyGluTyr and incubated for 30 minutes at room temperature. The plate was washed before incubating with the anti-phosphotyrosine-HRP antibody. The phosphorylation of the substrate is then reported quantitatively as the colorimetric read-out monitored by addition of ABTS. The results, shown in FIG. 3, exhibited that the polyGluTyr substrate was tyrosine phosphorylated by both chimeras although the Ret^{C634W}/Tie2^{ΔC} exhibited significantly greater activity than the Ret^{C634W}/Tie2^{WT}. Collectively, these results provide strong evidence that the cysteine substitution in the Ret extracellular domain is also a ligand-independent activating mutation for a heterologous kinase.

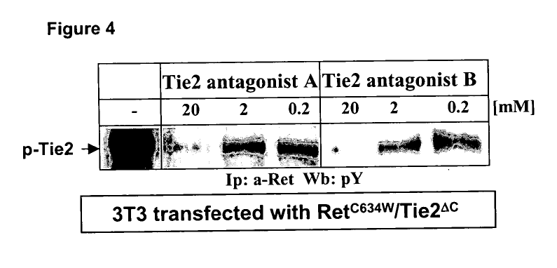

These ligand-independent chimera were utilized for formatting a biological assay for determining antagonists of the heterologous kinase. Under the same conditions, NIH 3T3 cells transfected with the Ret^{C634W}/Tie2^{ΔC} were treated with vehicle (DMSO), or small molecule Tie2 antagonists (OSI Pharmacueticals) at concentrations of 0.2, 2 or 20 μM for 2 hours at 37° C. before the cells were lysed. Following the immunoprecipitations, as described previously, the Tie2 kinase activity was determined by Western blotting with anti-phosphotyrosine-HRP. Shown in FIG. 4, autophosphotyrosine content of the Tie2 kinase in the Ret^{C634W}/Tie2^{ΔC} was inhibited by the antagonists, and the inhibition exhibited a dose dependent fashion. Thus, the chimeric receptor was capable of determining an antagonist of the heterologous kinase activated by the cysteine mutation in the Ret extracellular domain. To further develop a simple and large scale assay, a stable NIH 3T3 cell line expressing Ret^{C634W}/Tie2^{ΔC} was established by G418 selection. The stable cell line was seeded in a 96-well plate, and then treated with Tie2 antagonist for 2 hours at 37° C. The cells were lysed with cold TGH buffer, and the lysates were transferred to a Microlite2 plate pre-coated with anti-Ret monoclonal antibody, and incubated at 4° C. overnight. On the next day, the plate was washed and then incubated with the anti-phosphotyrosine-HRP for 2 hours at room temperature. The autophosphorylation was detected by addition of Super Signal ELISA Femto Maximum Sensitivity Substrate (Pierce) and chemiluminescence was read on a Wallac Victor² 1420 Multilabel Counter. IC50 of the compound was determined by an ExcelFit program. The IC50 curves (FIG. 6) of the Tie2 antagonist obtained from two independent assays were readily reproducible. Hence, the ligand-independent cysteine substitution in the Ret excellular domain provides a large scale method for screening antagonists of heterologous kinases under physiological conditions.

### Incorporation by Reference

All patents, published patent applications and other references disclosed herein are hereby expressly incorporated herein by reference.

### Equivalents

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A composition comprising a hybrid receptor protein-tyrosine kinase selected from:
(i) a cell comprising a hybrid receptor, wherein the hybrid receptor comprises (a) a modified extracellular domain of the Ret receptor kinase, containing one or more amino acid residue substitutions, deletions or additions that render it capable of activating an intracellular receptor kinase domain in a ligand-independent manner, and (b) the kinase domain of a heterologous receptor protein-tyrosine kinase, said kinase domain being rendered in an active conformation by its association with said Ret extracellular domain,
(ii) a membrane preparation isolated from a cell comprising a hybrid receptor, wherein the hybrid receptor comprises (a) a modified extracellular domain of the Ret receptor kinase, containing one or more amino acid residue substitutions, deletions or additions that render it capable of activating an intracellular receptor protein-tyrosine kinase domain in a ligand-independent manner, and (b) the kinase domain of a heterologous receptor protein-tyrosine kinase, said heterologous kinase domain being rendered in an active conformation by its association with said Ret extracellular domain, or
(iii) a hybrid receptor comprising (a) a modified extracellular domain of the Ret receptor kinase, containing one or more amino acid residue substitutions, deletions or additions that render it capable of activating an intracellular receptor protein-tyrosine kinase domain in a ligand-independent manner, and (b) the kinase domain of a heterologous receptor protein-tyrosine kinase, said heterologous kinase domain being rendered in an active conformation by its association with said Ret extracellular domain,

2. The composition of claim 1 wherein the kinase domain of the heterologous receptor protein-tyrosine kinase is selected from the kinase domains of EGFR, HER2, HER3, HER4, insulin receptor, IGF-1 receptor, IRR, PDGFR-alpha, PDGFR-beta, CSF-1 receptor, KIT, FLK2, FLK1, FLT4, FGFR1, FGFR2, FGFR3, FGFR4, CCK4, MET (HGF-R), RON, VEGFR1, VEGFR3, TrkA, Eph, AXL, MER, SKY, EphA2, EphA1, EphA3, EphA4, EphA5, EphA6, EphA7, EphA8, EphB1, EphB2, EphB3, EphB4, EphB5, EphB6, RYK, Flt-3, FLT-1, TRKC, TRKA, TRKB, Nck-alpha, Spry, KDR, PDGF-R-alpha, Syk, Blk, FGFR-3, LTK, TIE, Tie2, ROR, DDR1, DDR2, Ret, ROS, LTK, ALK, ROR2, ROR1, RTK106, LMR1, LMR2, LMR3, KLG, RYK, MuSK, LET-23, DAF-2, F59F3.1, F59F3.5, F40G9.13, EGL-15, KIN15, KIN16, TKR-16, TKR-1, C08H9.8, F59F5.3, M01B2.1, R09D1.12, R09D1.13, T01G5.1, T17A3.8, W04G5.6C, W04G5.6N, Y50D4B-4, ZK938.5, B0198.3, F54F7.5, VAB-1, C16B8.1, F11D5.3, C25F6.4, C16D9.2, CAM-1, T10H9.2, B0252.1, F11E6.8, F40A3.5, R151.4, T148.1, T22B11.3, Y38H6C.20, C24G6.2A, F08F1.1, F09A5.2, and F09G2.1.

3. The composition of claim 2 wherein the kinase domain of the receptor protein-tyrosine kinase is a human tie2 kinase domain.

4. The composition of claim 1 wherein the modified extracellular domain of the Ret receptor kinase comprises one or more amino acid residue substitutions, deletions or additions that result in one or more unpaired cysteine residues being available for Ret dimer formation.

5. The composition of claim 1 wherein the modified extracellular domain of the Ret receptor kinase comprises one or more amino acid residue substitutions at residues selected from Cys 609, Cys611, Cys618, Cys620, Cys630 and Cys634.

6. The composition of claim 1 wherein the modified extracellular domain of the Ret receptor kinase comprises one or more amino acid residue substitution selected from C634W, C634R, C634Y, C634F, C634G, C634S, C630F, C634W, C620F, C618F, C620S, C618S, C620G, C618G, C611G, C611W, C620R, C618R, C609R, C620Y, C618Y, C611Y, and C609Y.

7. The composition of claim 6 wherein the modified extracellular domain of the Ret receptor kinase comprises the amino acid residue substitution C634W.

8. The composition of claim 7 wherein the modified extracellular domain of the Ret receptor kinase comprises the extracellular domain of the human Ret receptor kinase with the amino acid residue substitution C634W.

9. The composition of claim 1 wherein the modified extracellular domain of the Ret receptor kinase comprises a deletion selected from L633, E632/L633 or residues 592-607.

10. The composition of claim 1 wherein the hybrid receptor has a transmembrane domain interposed between the modified extracellular domain of the Ret receptor kinase and the kinase domain of the heterologous receptor protein kinase.

11. The composition of claim 10, wherein the transmembrane domain comprises a transmembrane domain of a Ret receptor kinase.

12. The composition of claim 1 wherein the composition comprising a hybrid receptor protein-tyrosine kinase is a hybrid receptor comprising (a) a modified extracellular domain of the Ret receptor kinase, containing one or more amino acid residue substitutions, deletions or additions that render it capable of activating an intracellular receptor protein-tyrosine kinase domain in a ligand-independent manner, and (b) the kinase domain of a heterologous receptor protein-tyrosine kinase, said heterologous kinase domain being rendered in an active conformation by its association with said Ret extracellular domain.

13. A method for detecting a modulator of a selected receptor protein-tyrosine kinase, comprising
(a) providing a hybrid receptor comprising a modified extracellular domain of the Ret receptor kinase, containing one or more amino acid residue substitutions, deletions or additions that render it capable of activating an intracellular receptor protein-tyrosine kinase domain in a ligand-independent manner, and the heterologous kinase domain of the selected receptor protein-tyrosine kinase;
(b) incubating the hybrid receptor with a test sample;
(c) detecting a change in activity of the receptor protein-tyrosine kinase; and
(d) correlating said change with the presence of the modulator in the test sample.

14. The method of claim 13 wherein the change in activity of the receptor protein-tyrosine kinase is monitored by autophosphorylation of the hybrid receptor protein kinase, or by its activity on a peptide or protein substrate.

15. The composition of claim 1 wherein the composition comprising a hybrid receptor protein-tyrosine kinase is a cell comprising a hybrid receptor, wherein the hybrid receptor comprises (a) a modified extracellular domain of the Ret receptor kinase, containing one or more amino acid residue substitutions, deletions or additions that render it capable of activating an intracellular receptor kinase domain in a ligand-independent manner, and (b) the kinase domain of a heterologous receptor protein-tyrosine kinase, said kinase domain being rendered in an active conformation by its association with said Ret extracellular domain.

16. The composition of claim 15, wherein the cell is a eukaryotic cell.

17. The composition of claim 15, wherein the cell is a mammalian cell.

18. The composition of claim 15, wherein the cell is a human cell.

19. The composition of claim 15, wherein the cell is an insect cell.

20. The composition of claim 15, wherein the cell is a yeast cell.

21. A method for detecting a modulator of a selected receptor protein-tyrosine kinase, comprising
(a) providing a cell comprising a hybrid receptor, wherein the hybrid receptor comprises a modified extracellular domain of the Ret receptor kinase, containing one or more amino acid residue substitutions, deletions or additions that render it capable of activating an intracellular receptor protein-tyrosine kinase domain in a ligand-independent manner, and the heterologous kinase domain of the selected receptor protein-tyrosine kinase;
(b) incubating the cell with a test sample;
(c) detecting a change in activity of the receptor protein-tyrosine kinase; and
(d) correlating said change with the presence of the modulator in the test sample.

22. The method of claim 21 wherein the change in activity of the receptor protein-tyrosine kinase is monitored by autophosphorylation of the hybrid receptor protein kinase, or by its activity on a peptide or protein substrate.

23. The composition of claim 1 wherein the composition comprising a hybrid receptor protein-tyrosine kinase is a membrane preparation isolated from a cell comprising a hybrid receptor, wherein the hybrid receptor comprises (a) a modified extracellular domain of the Ret receptor kinase, containing one or more amino acid residue substitutions, deletions or additions that render it capable of activating an intracellular receptor protein-tyrosine kinase domain in a ligand-independent manner, and (b) the kinase domain of a heterologous receptor protein-tyrosine kinase, said heterologous kinase domain being rendered in an active conformation by its association with said Ret extracellular domain,

24. A method for detecting a modulator of a selected receptor protein-tyrosine kinase, comprising
(a) providing a membrane preparation comprising a hybrid receptor, wherein the hybrid receptor comprises a modified extracellular domain of the Ret receptor kinase, containing one or more amino acid residue substitutions, deletions or additions that render it capable of activating an intracellular receptor protein-tyrosine kinase domain in a ligand-independent manner, and the heterologous kinase domain of the selected receptor protein-tyrosine kinase;
(b) incubating the membrane preparation with a test sample;
(c) detecting a change in activity of the receptor protein-tyrosine kinase; and
(d) correlating said change with the presence of the modulator in the test sample.

25. The method of claim 24 wherein the change in activity of the receptor protein-tyrosine kinase is monitored by autophosphorylation of the hybrid receptor protein kinase, or by its activity on a peptide or protein substrate.

26. A nucleic acid encoding a hybrid receptor comprising (a) a modified extracellular domain of the Ret receptor kinase, containing one or more amino acid residue substitutions, deletions or additions that render it capable of activating an intracellular receptor protein-tyrosine kinase domain in a ligand-independent manner, and (b) the kinase domain of a heterologous receptor protein-tyrosine kinase, said heterologous kinase domain being rendered in an active conformation by its association with said Ret extracellular domain.

27. The nucleic acid of claim 26, wherein the nucleic acid is DNA.

28. A vector comprising the nucleic acid of claim 26.

29. A vector of claim 28 adapted for expression in a cell which vector comprises the regulatory elements necessary for expression of the nucleic acid in the cell operatively linked to the nucleic acid encoding the receptor so as to permit expression thereof.

30. The vector of claim 28, wherein the vector is a plasmid.

31. A host cell comprising the vector of claim 28.

32. The cell of claim 31, wherein the cell is a eukaryotic cell, a mammalian cell, a human cell, an insect cell, a yeast cell, or a prokaryotic cell.

33. A method for producing a hybrid receptor comprising (a) a modified extracellular domain of the Ret receptor kinase, containing one or more amino acid residue substitutions, deletions or additions that render it capable of activating an intracellular receptor protein-tyrosine kinase domain in a ligand-independent manner, and (b) the kinase domain of a heterologous receptor protein-tyrosine kinase, said heterologous kinase domain being rendered in an active conformation by its association with said Ret extracellular domain, said method comprising growing a host cell comprising the vector of claim 29 under suitable conditions permitting production of said hybrid receptor, and recovering the hybrid receptor.

34. The method of claim 33, further comprising preparing from the recovered hybrid receptor, a membrane preparation containing the hybrid receptor.

35. The method of claim 33, further comprising purifying the recovered hybrid receptor.
